(19) United States
(12) Patent Application Publication
(10) Pub. No.: US 2015/0133663 A1
(43) Pub. Date: May 14, 2015

(54) NOVEL SYNTHESIS METHOD

(71) Applicant: GE HEALTHCARE LIMITED, BUCKINGHAMSHIRE (GB)

(72) Inventors: Radha Achanath, Bangalore (IN); Jinto Jose, Bangalore (IN); Chitralekha Rangaswamy, Bangalore (IN); Subrata Mandal, Bangalore (IN); Afsal Mohammed Kadivilpparampu Mohamed, Bangalore (IN); Ian Martin Newington, Buckinghamshire (GB); Srinath Balaji, Karnataka (IN)

(21) Appl. No.: 14/344,181

(22) PCT Filed: Sep. 21, 2012
(86) PCT No.: PCT/EP2012/068662

§ 371 Date: Mar. 11, 2014

Related U.S. Application Data
(60) Provisional application No. 61/537,601, filed on Sep. 22, 2011.

(30) Foreign Application Priority Data
Sep. 22, 2011 (GB) 1116359.9

(51) Int. Cl. C07D 213/75

(52) U.S. Cl. CPC C07D 213/75
USPC 544/360

(57) ABSTRACT

The present invention relates to a method of making compounds having affinity for the 1 A subtype of the serotonin receptor, i.e. 5HT_{1A}. The method of the present invention provides advantages over the known methods of synthesis. The compounds obtained by the method of the invention have use in therapeutic methods. The compounds of the invention may also optionally compose a moiety suitable for detection by an in vivo imaging procedure and as such these compounds have use in in vivo imaging methods. The compounds have particular use in the treatment and diagnosis of various neurological and/or psychiatric disorders.

### TECHNICAL FIELD OF THE INVENTION

The present invention relates to a method of making compounds having affinity for the 1A subtype of the serotonin receptor, i.e. 5HT_{1A}. The method of the present invention provides advantages over the known methods of synthesis. The compounds obtained by the method of the invention have use in therapeutic methods. The compounds of the invention may also optionally comprise a moiety suitable for detection by an in vivo imaging procedure and as such these compounds have use in in vivo imaging methods. The compounds have particular use in the treatment and diagnosis of various neurological and/or psychiatric disorders.

### DESCRIPTION OF RELATED ART

Serotonin (5-hydroxytryptamine; 5HT) plays a role in several neurological and psychiatric disorders. It has been linked with major depression, bipolar disorder, eating disorders, alcoholism, pain, anxiety, obsessive-compulsive disorders, Alzheimer's disease (AD), Parkinson's disease (PD) and other psychiatric maladies. It is also involved in mediating the action of many psychotropic drugs including antidepressants, antianxiety drugs and antipsychotics. There are more than a dozen known subtypes of serotonin receptors. Among these serotonin receptors, 5HT_{1A} receptors play a role as a presynaptic autoreceptor in the dorsal raphe nucleus and as a Postsynaptic receptor for 5HT in terminal field areas. The serotonin system in the brain is an important neurotransmission network regulating various physiological functions and behaviour including anxiety and mood states. (See Rasmussen et al Chapter 1 “Recent Progress in Serotonin 5HT_{1A} Receptor Modulators”, in Annual Reports in Medicinal Chemistry, Vol. 30, Section I, pp. 1-9, 1995, Academic Press, Inc.).

Imaging the 5HT_{1A} receptor would be very useful in diagnosis or therapy monitoring of many CNS diseases including but not limited to AD (neuronal loss) and major depressive disorder (MDD). A relatively new antagonist tracer for positron emission tomography (PET) is trans-[¹⁸F]MeFWAY (Saigal et al 2006 J Nuc Med; 47: 1697), which is a promising tracer that has been suggested for application in AD diagnosis (Mukherjee et al 2006 J Lab Comp Radiopharm, 50: 375). The synthesis of the reference compound and its radiolabelling precursor is described by Mukherjee et al (2006 J Lab Comp Radiopharm; 50: 375) and a modified synthesis has recently been described by Choi et al (2010 Bull Chem Soc Korea; 31. 2371). Scheme 1 below illustrates the key steps of these prior art methods:

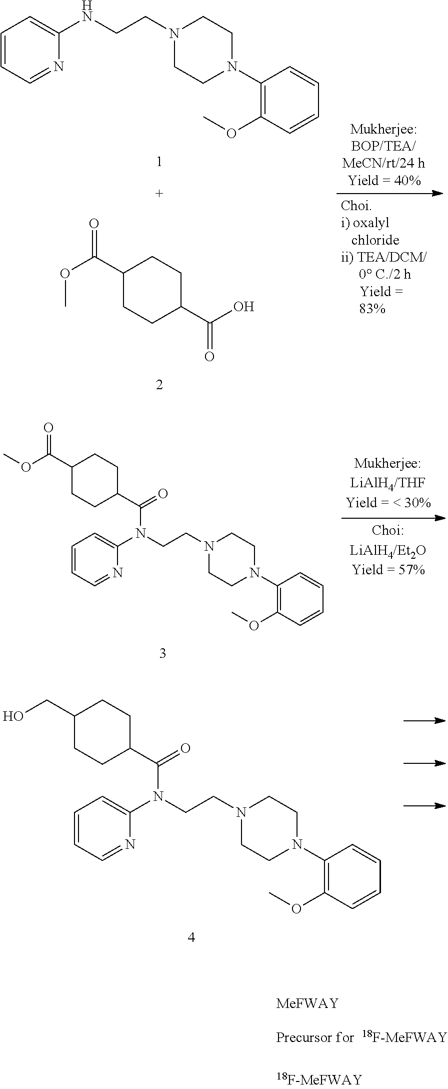 [SMILES: COc1ccccc1N1CCN(CCNc2ccccn2)CC1 ; "MeFWAY" ; COC(=O)C1CCC(C(=O)O)CC1 ; COC(=O)C1CCC(C(=O)N(CCN2CCN(c3ccccc3OC)CC2)c2ccccn2)CC1 ; COc1ccccc1N1CCN(CCN(C(=O)C2CCC(CO)CC2)c2ccccn2)CC1]

While there are advantages of the method of Choi et al over that of Mukherjee et al, not all of the purported advantages of the method of Choi et al have been reproducible in the hands of the present inventors. In particular, when the present inventors have tried to carry out the method of Choi et al on a slightly larger scale, difficulties have been encountered. The present inventors have observed that reduction from compound 3 to 4 still results in significant cleavage of the amide bond in addition to reduction of the ester. Cleavage of the amide bond has been found by the present inventors to be most pronounced when the reaction is scaled up, where the present inventors can find no advantage of the reduction method of Choi et al over the method of Mukherjee et al. Furthermore, due to its reduced thermodynamic stability, synthesis of cis-MeFWAY by following the prior art methods as described above for trans-MeFWAY is affected by multiple issues including epimerization during base hydrolysis, cleavage of the amide during LiAlH₄ reduction, and incomplete conversion from the ester to the alcohol.

Consequently there is scope for improved methods for the synthesis of MeFWAY and related compounds.

### SUMMARY OF THE INVENTION

The present invention provides a novel method for the preparation of MeFWAY and analogous compounds that provides advantages over the known methods. The synthetic route of this invention reduces the overall number of steps needed to prepare the compounds and uses milder reaction conditions. It is also amenable to scale-up. Furthermore, the method of the invention is suitable for obtaining respectable yields of the thermodynamically less stable cis-isomer.

### DETAILED DESCRIPTION OF THE INVENTION

### Method of Synthesis

In one aspect the present invention relates to a method of making a compound of Formula I:

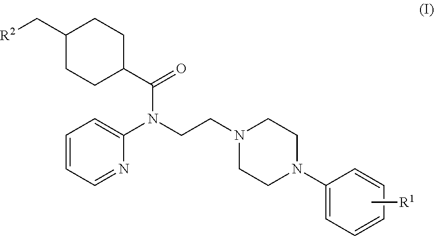

wherein:
R¹ is hydrogen, hydroxy, halogen or C₁₋₄ alkoxy;
R² is hydrogen, fluoro, bromo, chloro, C₁₋₄ alkyl, or is a leaving group;
wherein said compound optionally comprises one atom detectable in an in vivo imaging method;
or a pharmaceutically acceptable salt thereof, wherein said method comprises:
(i) borane reduction of a compound of Formula II:

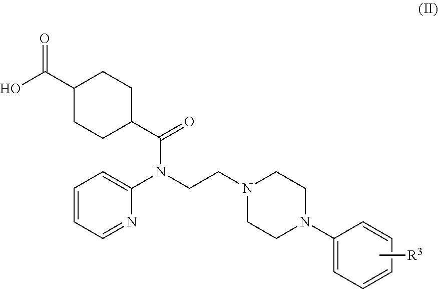

wherein R³ is as defined for R¹,
to obtain a compound of Formula III:

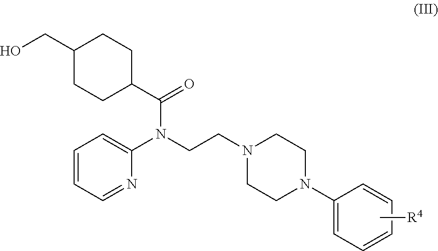

wherein R⁴ is as defined for le
(ii) conversion of said compound of Formula III to obtain said compound of Formula I.

The term “halogen” means a substituent selected from fluorine, chlorine, bromine or iodine as is intended to encompass radioactive as well as non-radioactive isotopes of these atoms. In particular, radioactive halogen atoms that may be detected by means of positron emission tomography (PET) or single-photon emission tomography (SPECT) are encompassed. For PET, suitable radioactive halogens are positron emitters and include ¹⁷F, ¹⁸F, ⁷⁵Br, ⁷⁶Br and ¹²⁴ I, wherein ¹⁸F and ¹²⁴I are preferred and ¹⁸F most preferred. For SPECT, suitable radioactive halogens are gamma emitters and include ¹²³I, ¹³¹I or ⁷⁷Br, with ¹²³I being preferred. ¹²⁵I is specifically excluded as it is not regarded as suitable for use in in vivo imaging.

The term “alkyl” as used herein means a radical having the general formula CₙH₂ₙ₊₁ wherein n is preferably an integer from 1-3. Examples of such radicals include methyl, ethyl, and isopropyl.

The term “alkoxy” means an alkyl as defined above which includes an ether radical in the chain (i.e. the group —O—) such as methoxy and ethoxy.

The term “fluoro” means a substituent that is either a radioactive isotope of fluorine, as defined above in connection with the definition of halogen, or a non-radioactive isotope of fluorine.

The term “bromo” means a substituent that is either a radioactive isotope of bromine, as defined above in connection with the definition of halogen, or a non-radioactive isotope of bromine.

The term “chloro” in the context of the present invention refers to a substituent that is a non-radioactive isotope of chlorine.

The teen “leaving group” refers to a moiety suitable for nucleophilic substitution and is a molecular fragment that departs with a pair of electrons in heterolytic bond cleavage. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

An “atom detectable in an in vivo imaging method” generally refers to any atom that can be detected external to a subject following administration to said subject as part of an in vivo imaging agent. In the case of the present invention it is contemplated that this atom is a radioactive isotope of an atom included in the definition for Formula I that may be detected by means of positron emission tomography (PET) or single-photon emission tomography (SPECT). Certain radioactive halogen atoms have already been defined above as suitable in this regard. In addition, it is envisaged that the compound of Formula I may comprise ¹¹C as the atom detectable in an in vivo imaging method, as ¹¹C is a useful positron-emitting isotope for PET imaging.

In the term “pharmaceutically acceptable salt” refers to a salt selected from (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycolic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine.

The term “borane reduction” refers to a reduction reaction carried out by means of a reagent comprising borane (BH₃) in a suitable form. Non-limiting examples of suitable reagents include diborane (B₂H₆) or a Lewis acid-Lewis base complex of BH₃. Examples of suitable Lewis acid-Lewis base complexes of BH₃ include BH₃.THF (tetrahydrofuran), or BH₃.Me₂S (dimethylsulfide).

The step of “conversion” of the compound of Formula III into the compound of Formula I refers to those synthetic steps required in order to add the desired substituents at either R¹ or R² of Formula I. Preferably in the context of the present invention modifications are carried out in order to introduce the desired substituent at R² of Formula I.

It is possible for the compounds defined in the context of the method of the invention to have one or more chiral centres and as such the compounds can exist in various stereoisomeric forms. Accordingly, the compounds of Formulae I-III are understood to encompass all possible stereoisomers. For example, in one embodiment the compounds of Formulae I-III may be of the following formulae, respectively:

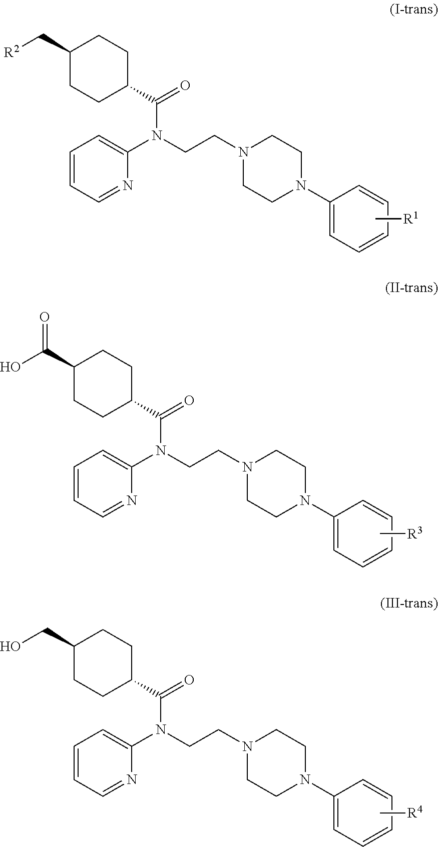

In another embodiment, the compounds of Formulae I-III may be of the following formulae, respectively:

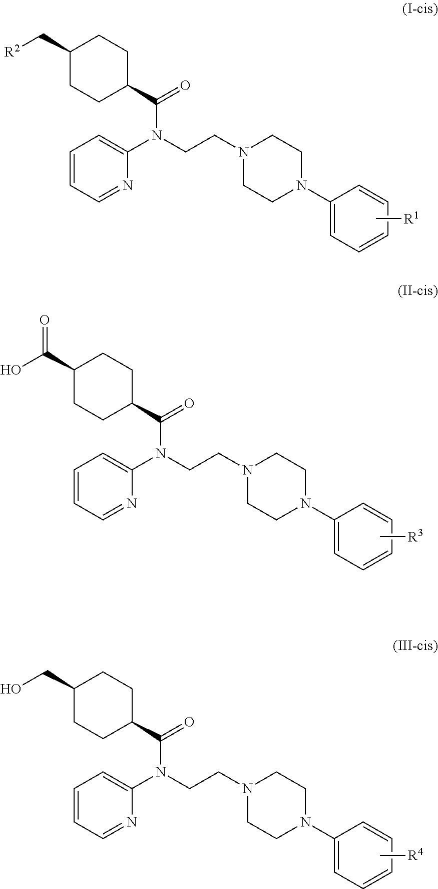

In Formula I R¹ is preferably hydroxyl, or alternatively preferably methoxy.

In Formula I, R² is preferably fluoro, wherein fluoro is preferably ¹⁸F. In an alternative, R² is preferably a leaving group as defined above, which results in a precursor compound suitable for obtaining said compound of Formula I wherein R² is ¹⁸F.

Compounds of Formula II for use in the method of the present invention may be prepared by use of or by straightforward adaptation of the methods described by Choi et al (2010 Bull Korean Chem Soc; 31(8): 2371-2374). Accordingly, reaction of 2-aminopyridine 1 with chloroacetyl chloride 2 at room temperature provides the 2-(chloroacetyl)amidopyridine 3:

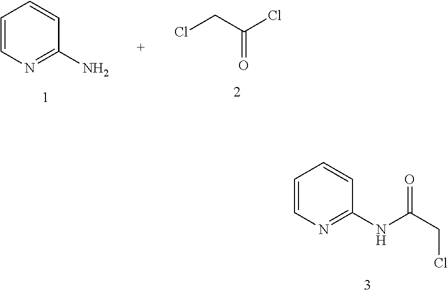 [SMILES: Nc1ccccn1 ; O=C(Cl)CCl ; O=C(CCl)Nc1ccccn1]

In the next step 3 is treated with the phenylpiperzine 4 in DMF at 80° C. in the presence of K₂CO₃ and NaI to give the corresponding phenylpiperazinyl amidopyridine 5:

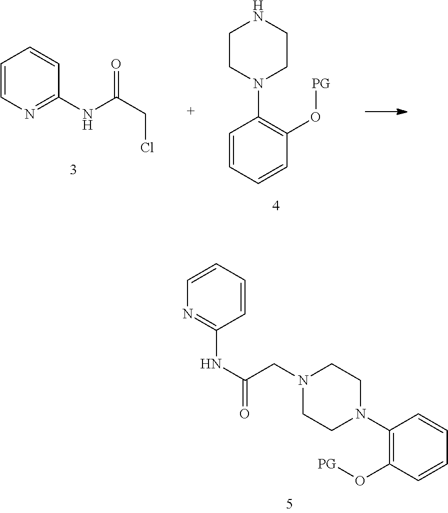

wherein PG represents hydrogen or a protecting group and is preferably a protecting group. A suitable protecting group is a methoxyethoxymethyl (MEM) group, a methoxymethyl (MOM) group, a t-butyldimethylsilyl (TBDMS) group, a trimethylsilyl (TMS) group or a benzyl group such as 4-methoxybenzyl or 2,4-dimethoxybenzyl.

Intermediate 5 where PG is hydrogen might alternatively be arrived at by first making the methylated derivative according to the method of Choi et al (supra), i.e. where PG of the above formula represents methyl, and demethylating to arrive at 5, and adding a suitable protecting group as defined above if desired. Non-limiting examples of reagents that can be used for this demethylation include BBr₃, trimethylsilyliodide, pyridinium tosylate and potassium t-butylthiolate.

5 is then reduced to obtain 6, a derivative of the known selective antagonist for 5HT1a receptors, WAY-100634:

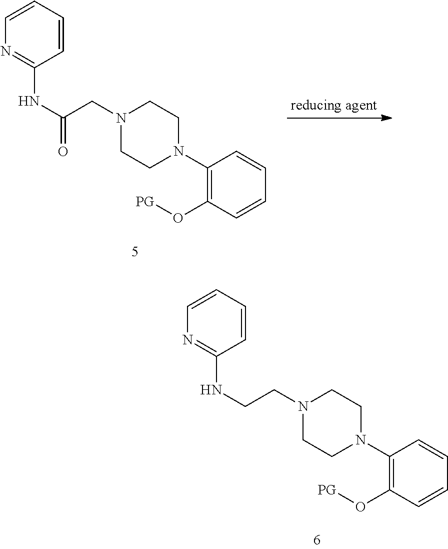

Alternatively, intermediate 6 might be arrived at by reduction of the methylated derivative of intermediate 5 (i.e. wherein PG is methyl) to remove the amide oxygen resulting in the methylated version of intermediate 6 (i.e. wherein PG is methyl), and then demethylating this product to obtain intermediate 6 wherein PG is hydrogen. A protecting group PG can be added using known methods if desired. Non-limiting examples of suitable means to carry out the reduction and demethylation (i.e. wherein PG is methyl) steps are as described elsewhere herein.

Using coupling conditions such as those described in Choi et al (2010 Bull Chem Soc Korea; 31: 2371) 6 can be coupled with cyclohexane-1,4-dicarboxylic acid 9 to lead to carboxylic acid intermediate 10, a compound of Formula II, after aqueous work-up:

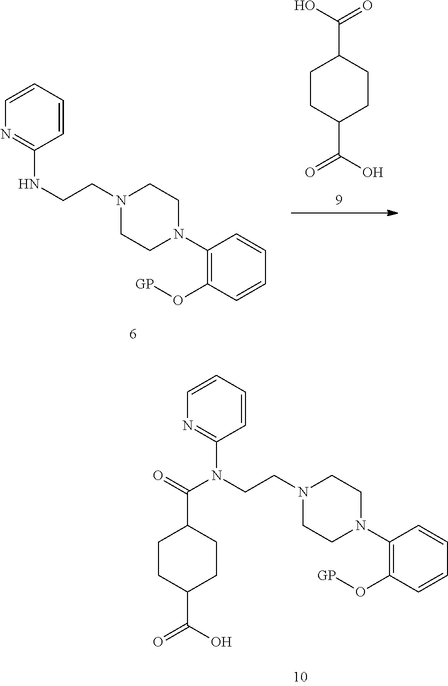

Using the symmetrical di-acid compound 9 provides an additional advantage over the prior art methods where 4-carbomethoxycyclohexane-1-carboxylic acid is used in the coupling step, which requires preparation from 9 and subsequent purification before use. This preferred aspect of the invention therefore results in a method which requires even less steps than the prior art methods.

Reduction of 10 with a borane reducing agent gives 12, a compound of Formula III. An advantage is provided over known methods as this reducing agent does not result in the unwanted production of any significant amounts of amide cleavage, which regenerates 6. Further, the method of the present invention allows scaling up of the production of compounds of Formula I to quantities that the present inventors have found are not permitted by the prior art methods. Therefore, the method of the present invention allows production of compounds of Formula I, for example from 100 mg up to gram quantities, from 200 mg to gram quantities, or from 500 mg to gram quantities. The term “gram quantities” is taken to mean at least 1 g.

Also, in the case of the cis-isomer there is an even more marked advantage with using borane reduction. When LiAlH₄ is used as the reducing agent, as in the prior art methods, it converts to basic lithium hydroxide as soon as it contacts water. The present inventors have observed that cis to trans isomerization of the compounds described herein is triggered under basic conditions. It is particularly desirable therefore that the borane reduction step is mildly acidic. Examples of preferred borane reducing agents include borane-THF complex and borane-dimethyl sulfide.

Alternatively, 6 can be reacted with 11 to give 12 directly using an amide coupling reagent. Non-limiting examples of suitable coupling agents include dicyclohexyl carbodiimide, 2-(1H-7-Azabenzotriazol-1-yl)-1,1,3,3-tetramethyl uronium hexafluorophosphate Methanaminium (HATU), benzotriazol-1-yl-oxytripyrrolidinophosphonium hexafluorophosphate (PyBOP), or other benzotriazole-based peptide coupling reagents:

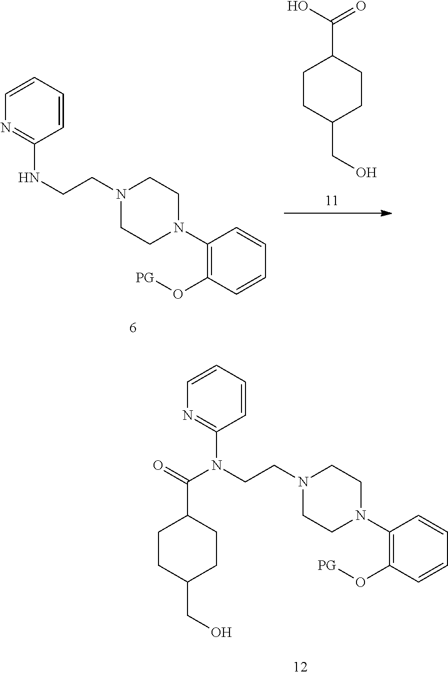

Intermediate 12 can then be converted using known methods, and subsequently deprotected where PG is a protecting group, to obtain compounds of the present invention, e.g.

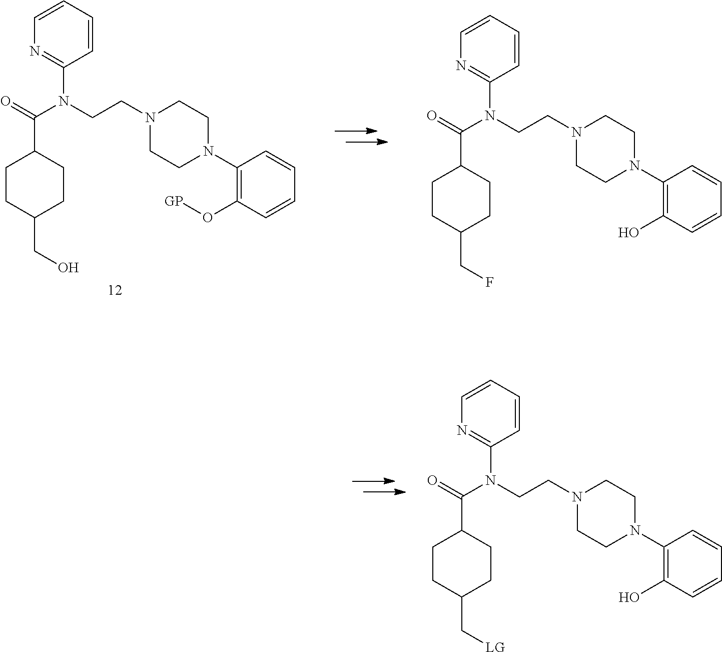

wherein LG is a leaving group as defined hereinabove.

The compound of Formula III can alternatively be regarded as a product. Therefore, in another aspect, the present invention relates to a method of making said compound of Formula III comprising the borane reduction step (i) as defined above. Any aspects of the invention described herein for the method of making a compound of Formula I that are applicable to the method of making said compound of Formula III apply equally to said latter method.

In a preferred embodiment, the conversion step of the present invention comprises reaction of said compound of Formula III with a suitable source of fluorine, bromine or chlorine to obtain a compound of Formula I wherein R² is fluoro, bromo or chloro. Suitable sources of fluorine, bromine or chlorine are well-known to the person skilled in the art are readily available.

In an alternative preferred embodiment, the conversion step of the present invention comprises reaction of said compound of Formula III with a suitable source of a leaving group to obtain a compound of Formula I wherein R² is a leaving group. In this embodiment of the invention, the method comprises the further step of reacting said compound of Formula I wherein R² is a leaving group with a suitable source of ¹⁸F to obtain a compound of Formula I wherein R² is ¹⁸F. The “suitable source of ¹⁸F” preferably refers to [¹⁸F]fluoride.

[¹⁸F]fluoride (¹⁸F⁻) for radiofluorination reactions is normally obtained as an aqueous solution from the nuclear reaction ¹⁸O(p,n)¹⁸F and is made reactive by the addition of a cationic counterion and the subsequent removal of water. A suitable cationic counterion for this purpose should possess sufficient solubility within the anhydrous reaction solvent to maintain the solubility of ¹⁸F⁻. Suitable counterions include large but soft metal ions such as rubidium or caesium, potassium complexed with a cryptand such as Kryptofix™, or tetraalkylammonium salts. A preferred suitable source of [¹⁸F]fluoride is selected from [¹⁸F]potassium fluoride and [¹⁸F]caesium fluoride, most preferably [¹⁸F]potassium fluoride wherein Kryptofix™ is used to activate the [¹⁸F]fluoride ion because of its good solubility in anhydrous solvents and enhanced ¹⁸F⁻ reactivity.

The synthesis of ¹⁸F-labelled compounds, particularly for use as PET tracers, is currently most conveniently carried out by means of an automated synthesis apparatus, e.g. Tracerlab™ and FASTlab™ (both GE Healthcare). In a preferred embodiment, the method to obtain the ¹⁸F-labelled compound Formula I is automated, preferably via an automated synthesis apparatus. The radiochemistry is performed on the automated synthesis apparatus by fitting a “cassette” to the apparatus. Such a cassette normally includes fluid pathways, a reaction vessel, and ports for receiving reagent vials as well as any solid-phase extraction cartridges used in post-radiosynthetic clean up steps. The reagents, solvents and other consumables required for the automated synthesis may also be included together with a data medium, such as compact disc carrying software, which allows the automated synthesiser to be operated in a way to meet the end user's requirements for concentration, volumes, time of delivery etc.

In a further preferred embodiment, the method of the present invention further comprises formulation of the compound of Formula I (apart from wherein R² is a leaving group) to obtain a pharmaceutical composition comprising said compound and a physiologically acceptable carrier or vehicle.

The pharmaceutical composition can be administered orally or by any other convenient route, for example, by infusion or bolus injection, or by absorption through epithelial or mucocutaneous linings (e.g., oral, rectal, and intestinal mucosa, etc. Administration can be systemic or local. Various delivery systems suitable for administration to a subject are known, e.g., encapsulation in liposomes, microparticles, microcapsules, capsules, etc.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the compound of the present invention into the bloodstream.

The pharmaceutical composition can optionally comprise a suitable amount of a physiologically acceptable excipient so as to provide the form for proper administration of the composition to a subject. Such a physiologically acceptable excipient can be a liquid, such as water for injection, bactereostatic water for injection, sterile water for injection, and oils, including those of petroleum, subject, vegetable, or synthetic origin, such as peanut oil, soybean oil, mineral oil, sesame oil and the like. The pharmaceutical excipients can be saline, gum acacia; gelatine, starch paste, talc, keratin, colloidal silica, urea and the like. In addition, auxiliary, stabilizing, thickening, lubricating, and colouring agents can be used. In one embodiment the physiologically acceptable excipients are sterile when administered to a subject. Water is a particularly useful excipient when the compound of the present invention is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. The pharmaceutical composition, if desired, can also contain minor amounts of wetting or emulsifying agents, or pH buffering agents. The pharmaceutical composition can take the form of solutions, suspensions, emulsion, tablets, pills; pellets, capsules, capsules containing liquids, powders, sustained-release formulations, suppositories, emulsions. aerosols, sprays, suspensions, or any other form suitable for use.

The present invention is illustrated by the following non-limiting examples.

### BRIEF DESCRIPTION OF THE EXAMPLES

Example 1 describes the synthesis of (1r,4r)-4-(fluoromethyl)-N-(2-(4-(2-methoxyphenyl)piperazin-1-yl)ethyl)-N-(pyridin-2-yl)cyclohexanecarboxamide (trans-MeFWAY).

Example 2 describes the synthesis of (1r,4r)-4-(fluoromethyl)-N-(2-(4-(2-((2-methoxyethoxy)methoxy)phenyl)piperazin-1-yl)ethyl)-N-(pyridin-2-yl)cyclohexanecarboxamide.

Example 3 describes the synthesis of (1r,4r)-4-([¹⁸F]fluoromethyl)-N-(2-(4-(2-hydroxyphenyl)piperazin-1-yl)ethyl)-N-(pyridin-2-yl)cyclohexanecarboxamide.

Example 4 describes the synthesis of (1s,4s)-4-(fluoromethyl)-N-(2-(4-(2-methoxyphenyl)piperazin-1-yl)ethyl)-N-(pyridin-2-yl)cyclohexanecarboxamide

Example 5 is a comparative example describing a prior art reduction of (1s,4s-methyl 4-((2-(4-(2-methoxyphenyl)piperazin-1-yl)ethyl)-N-(pyridin-2-yl)cyclohexanecarboxamide to (1s,4s)-4-(hydroxymethyl)-N-(2-(4-(2-methoxyphenyl)piperazin-1-yl)ethyl)-N-(pyridin-2-yl)cyclohexanecarcoxamide.

### LIST OF ABBREVIATIONS USED IN THE EXAMPLES

Boc tert-Butyloxycarbonyl
DAST Diethylaminosulfur trifluoride

### DCM Dichloromethane

DMF Dimethyl formamide
LC-MS liquid chromatography-mass spectrometry

### MEM 2-Methoxyethoxymethyl

NMR nuclear magnetic resonance

### OTs Tosylate

PG protecting group
TEA Triethyl amine
TFA Trifluoroacetic acid

### Example 1

### Synthesis of (1r,4r)-4-(fluoromethyl)-N-(2-(4-(2-methoxyphenyl)piperazin-1-yl)ethyl)-N-(pyridin-2-yl)cyclohexanecarboxamide (MeFWAY)

### 1(i) 2-chloro-N-(pyridin-2-yl)acetamide

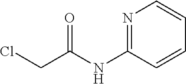 [SMILES: O=C(CCl)Nc1ccccn1]

To a solution of 2-aminopyridine (2 g, 21.3 mmol) and TEA (3.23 g, 31.9 mmol, 4.4 mL) in anhydrous DCM (20 mL) was slowly added chloroacetyl chloride (3.96 g, 35.1 mmol, 2.8 mL) at 0° C. The reaction mixture was stirred at room temperature under a nitrogen atmosphere for 18 h. The reaction mixture was partitioned between DCM (50 mL) and water (50 mL); the organic portion was dried (phase separation cartridge) and evaporated to dryness to afford a brown oil.

The residue was purified by column chromatography on silica gel eluting with petroleum ether (A): ethyl acetate (B) (15-50% (B), 40 g, 10.0 CV, 40 mL/min) to afford a beige solid (2.31 g, 64%). The ¹H NMR indicated presence of both starting materials so the product was re-purified by column chromatography on high performance silica gel eluting with petroleum ether (A): ethyl acetate (B) (40-75% (B), 40 g, 18.3 CV, 40 mL/min) to afford the product as a beige solid (1.92 g, 53%).

LC-MS: m/z calcd for C₇H₇ClN₂O, 170.0; found, 171.0 (M+H)⁺.

¹H NMR (300 MHz, CDCl₃): δ_{H} 4.18 (2H, s, CH₂), 7.06-7.10 (1H, in, pyridyl-5-CH), 7.68-7.75 (1H, in, pyridyl-4-CH), 8.17 (1H, d, J=8.3 Hz, pyridyl-3-CH), 8.30 (1H, dd, J=4.9 Hz and 1.0 Hz, pyridyl-6-CH) and 8.98 (1H, s, NH). ¹³C NMR (75 MHz, CDCl₃): δ_{c} 42.8 (CH₂), 1119 (pyridyl-3-CH), 120.5 (pyridyl-5-CH), 138.5 (pyridyl-4-CH), 147.9 (pyridyl-6-CH), 150.4 (pyridyl-2-CN) and 164.5 (C═O).

### 1(ii) 2-(4-(2-methoxyphenyl)piperazin-1-yl)-N-(pyridin-2-yl)acetamide

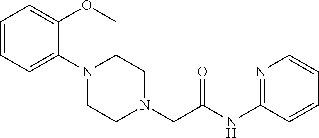 [SMILES: COc1ccccc1N1CCN(CC(=O)Nc2ccccn2)CC1]

To a solution of 1-(2-methoxyphenyl)piperazine (2.16 g, 11.25 mmol) in DMF (20 mL) was added potassium carbonate (3.89 g, 28.14 mmol) and was stirred at 80° C. for one hour. To the cooled reaction mixture was added a solution of 2-chloro-N-(pyridin-2-yl)acetamide (1.92 g, 11.25 mmol) in DMF (10 mL) and sodium iodide (253 mg, 1.69 mmol) and was stirred at 80° C. for 3 h. The cooled reaction mixture was partitioned between ethyl acetate (2*50 mL) and water (50 mL) and the organic portion was dried (MgSO₄), filtered and evaporated to dryness. The residue was purified by column chromatography on silica gel eluting with petroleum ether (A): ethyl acetate (B) (50-100% (B), 100 g, 27.0 CV, 60 mL/min) to afford the desired product as an off-white gum (2.81 g, 77%).

LC-MS m/z calcd for C₁₈H₂₂N₄O₂, 326.2; found, 327.0.

¹H NMR (300 MHz, CDCl₃): δ_{H} 2.82 (4H, t, J=4.8 Hz, 2′- & 6′-CH₂), 3.17 (4H, br s, 3′- & 5′-CH₂), 3.23 (2H, s, CH₂), 3.86 (3H, s, OCH₃), 6.85-7.06 (5H, m, 4× phenyl-CH and pyridyl-5-CH), 7.70 (1H, td, J=7.8 Hz and 1.9 Hz, pyridyl-4-CH), 8.24-8.32 (2H, m, pyridyl-3-CH and pyridyl-6-CH) and 9.63 (1H, s, NH). ¹³C NMR (75 MHz, CDCl₃): δ_{C} 50.6 (3′- & 5′-CH₂), 53.8 (4′- & 6′-CH₂), 55.3 (OCH₃), 62.2 (CH₂), 111.2 (phenyl-3-CH), 113.8 (pyridyl-3-CH), 118.3 (phenyl-5-CH), 119.8 (phenyl-4-CH), 121.0 (phenyl-6-CH), 123.1 (pyridyl-5-CH), 138.3 (pyridyl-4-CH), 140.9 (phenyl-2-C), 147.9 (pyridyl-6-C), 151.0 (pyridyl-2-C), 152.2 (phenyl-1-C) and 169.2 (C═O).

### 1(iii) N-(2-(4-(2-methoxyphenyl)piperazin-1-yl)ethyl)pyridin-2-amine

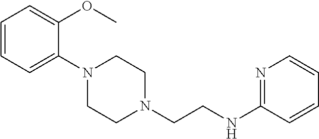 [SMILES: COc1ccccc1N1CCN(CCNc2ccccn2)CC1]

To a solution of 2-(4-(2-methoxyphenyl)piperazin-1-yl)-N-(pyridin-2-yl)acetamide (5.8 g, 17.8 mmol) in THF (80 mL) at 0° C. was slowly added LiAlH₄ (2.02 g, 53.3 mmol, 26.7 mL of a 2.0 M solution in THF) and was stirred at ambient temperature for three hours. The reaction mixture was cooled to 0° C. and quenched with saturated ammonium chloride solution (10 mL); this was then filtered with ethyl acetate and the resultant solution was partitioned between ethyl acetate (150 in L) and water (150 mL). The organic portion was dried (MgSO₄), filtered and evaporated to dryness to afford the desired product as a yellow oil (4.37 g, 79%).

LC-MS m/z calcd for C₁₈H₂₄N₄O, 312.2; found, 313.1.

¹H NMR (300 MHz, CDCl₃): δ_{H} 2.69 (6H, t, J=6.0 Hz, 2″-CH₂ and 2′- & 6′-CH₂), 3.10 (4H, br s, 3′- & 5′-CH₂), 3.37 (2H, q, J=5.8 Hz, 1″-CH₂), 3.86 (3H, s, OCH₃), 5.13 (1H, br s, NH), 6.41 (1H, d, J=8.6 Hz, pyridyl-5-CH), 6.57 (1H, ddd, J=7.0 Hz, 5.2 Hz and 0.9 Hz, pyridyl-5-CH), 6.84-7.02 (4H, m, 4× phenyl-CH), 7.41 (1H, ddd, J=8.4 Hz, 7.1 Hz and 1.9 Hz, pyridyl-4-CH) and 8.09 (1H, ddd, J=4.9 Hz, 1.8 Hz and 0.9 Hz, pyridyl-6-CH). ¹³C NMR (75 MHz, CDCl₃): δ_{C} 38.5 (1″-CH₂), 50.6 (3′- & 5′-CH₂), 53.1 (4′- & 6′-CH₂), 55.3 (OCH₃), 56.8 (2″-CH₂), 107.0 (pyridyl-3-CH), 111.1 (phenyl-3-CH), 112.6 (pyridyl-5-CH), 118.2 (phenyl-5-CH), 121.0 (phenyl-4-CH), 122.9 (phenyl-6-CH), 137.3 (pyridyl-4-CH), 141.3 (phenyl-2-C), 148.2 (pyridyl-6-CH), 152.2 (pyridyl-2-C) and 158.8 (phenyl-1-C).

### 1(iv) (1r,4r)-4-((2-(4-(2-methoxyphenyl)piperazin-1-yl)ethyl)(pyridin-2-yl)carbamoyl)cyclohexanecarboxylic acid

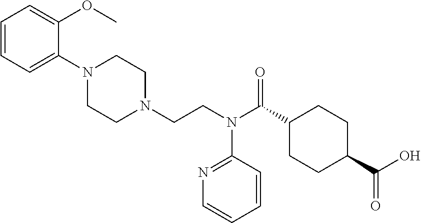

A mixture of trans 1,4-cyclohexanedicarboxlic acid (1 g, 5.813 mmol) and oxalyl chloride (7.4 g, 58.2 mmol, 5 mL) was heated to reflux for 1 h. The excess oxalyl chloride was co-distilled using dichloromethane under nitrogen atmosphere. The solid obtained was dissolved in DCM (50 mL). To the resulting mixture, a solution of N-(2-(4-(2-methoxyphenyl)piperazin-1-yl)ethyl)pyridin-2-amine (1.45 g, 4.65 mmol) and triethylamine (1.152 g, 11.4 mmol, 1.6 mL) in DCM (50 mL) was added slowly at 25° C. under nitrogen atmosphere. After the complete addition, the mixture was stirred at 25° C. for 1 h. The reaction mixture was quenched with water (20 mL) and the DCM layer separated and evaporated to obtain a residue. The residue was dissolved in a sodium hydroxide solution (1 g dissolved in 40 mL water) and the resulting aqueous layer was washed with DCM (25 mL×2). The aqueous layer was adjusted to a pH 6.5-6.6 (using cone HCl) and extracted with DCM (25 mL×2). The DCM layer was dried over Na₂SO₄ and evaporated to obtain the desired product as white foam (1.1 g, 52%).

LC-MS: m/z calcd for C₂₆H₃₄N₄O₄, 466.3; found, 466.2

¹H NMR (500 MHz, CDCl₃): δ_{H} 1.03-1.86 (10H, m, 6× cyclohexyl-CHH and CHC(═O)N), 2.67-2.87 (6H, m, 3′- & 5′-CH₂ and 2″-CH₂), 3.04 (4H, br s, 4′- & 6′-CH₂), 3.83 (3H, s, phenyl-OCH₃), 3.95 (2H, in, 1″-CH₂), 6.95-7.01 2(4H, m, 4× phenyl-CH), 7.20-7.32 (2H, m, pyridyl-3-CH, pyridyl-5-CH), 7.72-7.78 (1H, t, J=5 Hz, pyridyl-4-CH), and 8.52 (1H, d, J=5 Hz, pyridyl-6-CH)

### 1(v) (1r,4r)-4-(hydroxymethyl)-N-(2-(4-(2-methoxyphenyl)piperazin-1-yl)ethyl)-N-(pyridin-2-yl)cyclohexanecarboxamide

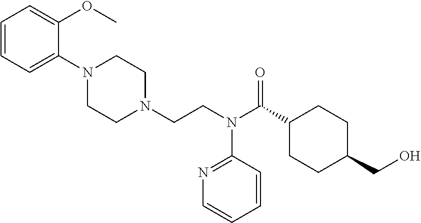

(1r,4r)-4-((2-(4-(2-methoxyphenyl)piperazin-1-yl)ethyl)(pyridin-2-yl)carbamoyl)cyclo-hexanecarboxylic acid (700 mg, 1.5 mmol) was dissolved in dry THF (15 mL) and cooled to 0° C. Borane-tetrahydrofuran complex (2.0 g, 23.25 mmol, 23.0 mL) was added to the cold solution in three equal lots, every 1 h. After the complete addition, the mixture was stirred at 25° C. for 1 h. The reaction mixture was quenched with water (1 mL) and THF evaporated. The residue obtained was dissolved in methanol (10 mL) and heated to reflux for 1 h. Evaporated methanol and the residue (containing high boiling) was co-distilled using hexane (100 mL×3) to obtain the crude product (0.65 g, 97%), which was used in the next step without further purification.

LC-MS: m/z calcd for C26H36N4O3, 452.3, found, 452.3

### 1(vi) ((1r,4r)-4-((2-(4-(2-methoxyphenyl)piperazin-1-yl)ethyl)(pyridin-2-yl)carbamoyl)cyclohexyl)methyl 4-methylbenzenesulfonate

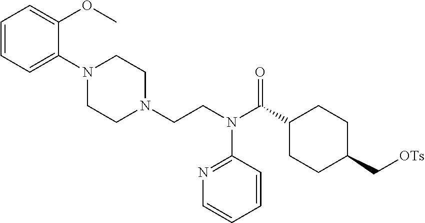

To a solution of (1r,4r)-4-(hydroxymethyl)-N-(2-(4-(2-methoxyphenyl)piperazin-1-yl)ethyl)-N-(pyridin-2-yl)cyclohexanecarboxamide (850 mg, 1.88 mmol) in DCM (10 mL) was added tosyl chloride (1 g, 5.2 mmol) and TEA (0.72 g, 7.12 mmol, 1 mL). The mixture was stirred at 25° C. for 24 h. The reaction mixture was quenched with 10% aqueous sodium bicarbonate solution (50 mL) and the DCM layer separated. The DCM layer was dried (Na₂SO₄) and evaporated to dryness. The residue was purified by manual column chromatography on neutral alumina (100 g) eluting with Hexane (A): Ethyl acetate (B) (10-50% (B), to afford the desired product as foam on drying under high vacuum (550 mg, 48%).

LC-MS: m/z calcd for C₃₃H₄₂N₄O₅S, 606.3; found, 605.6

1H NMR (300 MHz, CD₃CN): δH 0.71 (2H, q, J=12 Hz, 2× cyclohexyl-CHH), 1.34-1.83 (7H, in, 6× cyclohexyl-CHH and CHC(═O)N), 1.96 (1H, t, J=10.5 Hz, cyclohexyl-CHCH₂OTs), 2.44 (3H, s, tosyl-CH₃), 2.46-2.58 (6H, m, 3′- & 5′-CH₂ and 2″-CH₂), 2.90 (4H, br s, 4′- & 6′-CH₂), 3.75 (2H, d, J=6 Hz, CH₂OTs), 3.79 (3H, s, phenyl-OCH₃), 3.88 (2H, t, J=6.0 Hz, 1″-CH₂), 6.82-7.04 (4H, m, 4× phenyl-CH), 7.25-7.48 (4H, m, pyridyl-3-CH, pyridyl-5-CH and 2× tosyl-CHCCH₃), 7.68-7.88 (3H, m, pyridyl-4-CH and 2× tosyl-CHCSO₂) and 8.48 (1H, d, J=5 Hz, pyridyl-6-CH).

### 1(vii) (1r,4r)-4-(fluoromethyl)-N-(2-(4-(2-methoxyphenyl)piperazin-1-yl)ethyl)-N-(pyridin-2-yl)cyclohexanecarboxamide (trans-MeFWAY)

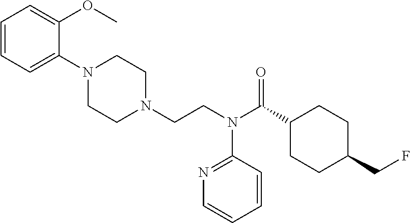

To a solution of (1r,4r)-4-(hydroxymethyl)-N-(2-(4-(2-methoxyphenyl)piperazin-1-yl)ethyl)-N-(pyridin-2-yl)cyclohexanecarboxamide (40 mg, 0.09 mmol) in DCM (2 mL) in an ice-water bath was added DAST (21 mg, 0.13 mmol, 17 uL) and was stirred at ambient temperature under a nitrogen atmosphere for 94 h. The reaction mixture was quenched with 10% aqueous sodium bicarbonate solution (10 mL) and partitioned between the aqueous and DCM (10 mL). The organic portion was dried (phase separation cartridge) and evaporated to dryness. The residue was purified by column chromatography on silica gel eluting with DCM (A): methanol (B) (2-10% (B), 4 g, 76.0 CV, 18 mL/min) to afford the desired product as a colourless oil (14 mg, 35%).

LC-MS m/z calcd for C₂₆H₃₅FN₄O₂, 454.3, found 455.2 (M+H)⁺

¹H NMR (300 MHz, CDCl₃): δ_{H} 0.83 (2H, q, J=11.7 Hz, 2× cyclohexyl-CHH), 1.54-1.86 (7H, m, 6× cyclohexyl-CHH and cyclohexyl-CHC(═O)N), 2.19 (1H, t, J=11.9 Hz, cyclohexyl-CHCH₂F), 2.61 (6H, m, 2× piperazinyl-CH₂ and 2″-CH₂), 2.98 (4H, br s, 2× piperazinyl-CH₂), 3.84 (3H, s, phenyl-OCH₃), 3.98 (211, t, J=6.9 Hz, 1″-CH₂), 4.15 (2H, dd, J_{CF}=47.7 Hz, J=5.4 Hz, CH₂F), 6.83-7.01 (4H, m, 4× phenyl-CH), 7.22-7.31 (2H, m, pyridyl-3-CH and pyridyl-5-CH), 7.76 (1H, td, J=7.7 Hz and 1.8 Hz, pyridyl-4-CH) and 8.52 (1H, dd, J=4.9 Hz and 1.2 Hz, pyridyl-6-CH). ¹³C NMR (75 MHz, CDCl₃): δ_{C} 27.4 (2× cyclohexyl-CH₂(CHCH₂F)), 28.7 (2× cyclohexyl-CH₂(CHC(═O)N)), 37.7 (cyclohexyl-CH(CH₂F), 42.1 (cyclohexyl-CHC(═O)N), 45.3 (1″-CH₂), 50.6 (2′- & 6′-CH₂), 53.4 (2″-, 3′- & 5′-CH₂), 55.3 (phenyl-OCH₃), 111.2 (phenyl-3-CH), 118.1 (phenyl-5-CH), 120.9 (phenyl-4-CH), 122.2 (phenyl-6-CH), 122.8 (pyridyl-5-CH and pyridyl-3-CH), 138.2 (pyridyl-4-CH), 142.3 (phenyl-2-CO), 149.3 (pyridyl-6-CH), 152.2 (phenyl-1-CN) and 175.8 (C═O). ¹⁹F NMR (283 MHz, CDCl₃): δ_{F} −223.9.

### Example 2

### Synthesis of (1r,4r)-4-(fluoromethyl)-N-(2-(4-(2-((2-methoxyethoxy) methoxy)phenyl)piperazin-1-yl)ethyl)-N-(pyridin-2-yl)cyclohexanecarboxamide

### 2(i) tert-butyl 4-(2-hydroxyphenyl)piperazine-1-carboxylate

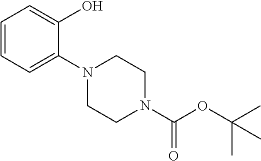 [SMILES: CC(C)(C)OC(=O)N1CCN(c2ccccc2O)CC1]

To a solution of 2-(1-piperazino)phenol (3.0 g, 16.8 mmol) and NaHCO₃ (2.12 g, 25.3 mmol) in a 1:1:1 mixture of THF/H₂O/dioxane (60 mL) was added Boc₂O (4.41 g, 20.2 mmol) and was stirred at ambient temperature for 20 mins until a solid formed. The reaction mixture was filtered and the filtrate was partitioned between water (100 mL) and DCM (100 mL); the organic portion was dried (phase separation cartridge) and evaporated to dryness. The combined residue and solid product were recrystallized from boiling petroleum ether to afford tert-butyl 4-(2-hydroxyphenyl)piperazine-1-carboxylate as a beige solid (3.38 g, 72%).

LC-MS: m/z calcd for C₁₅H₂₂N₂O₃, 278.2; found, 277.0 (M−H)⁺.

¹H NMR (301 MHz, CHLOROFORM-D) δ 7.14-7.05 (m, 2H, phenyl-3-CH and phenyl-4-CH), 6.98-6.93 (m, 1H, phenyl-6-CH), 6.89-6.83 (m, 1H, phenyl-5-CH), 3.63-3.53 (m, 4H, 2′- & 6′-CH₂), 2.87-2.77 (m, 4H, 3′- & 5′-CH₂), 1.50-1.48 (s, 9H, 3× CH₃).

### 2(ii) tert-butyl 4-(2-((2-methoxyethoxy)methoxy)phenyl)piperazine-1-carboxylate

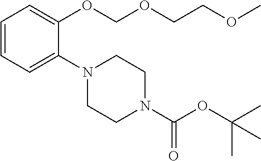 [SMILES: COCCOCOc1ccccc1N1CCN(C(=O)OC(C)(C)C)CC1]

To a solution of tert-butyl 4-(2-hydroxyphenyl)piperazine-1-carboxylate (3.30 g, 11.9 mmol) in DMF (100 mL) at 0° C. was slowly added sodium hydride (474 mg of a 60% dispersion in mineral oil, 11.9 mmol) and was stirred for 30 mins. Thereto was then added MEM-Chloride (1.48 g, 11.9 mmol, 1.35 mL) and was stirred at 60° C. for 18 h. The reaction mixture was evaporated to dryness and the residue was partitioned between ethyl acetate (2*75 mL) and water (75 mL). The organic portion was washed with brine (75 mL), dried over magnesium sulfate, filtered and evaporated to dryness. The residue was purified by column chromatography on silica gel eluting with petroleum ether (A): ethyl acetate (B) (10-40% (B), 50 g, 20.0 CV, 40 mL/min) to afford tert-butyl 4-(2((2-methoxyethoxy)methoxy)phenyl)piperazine-1-carboxylate as a colourless oil (937 mg, 22%).

¹H NMR (301 MHz, CHLOROFORM-D) δ 7.15-7.09 (m, 1H, phenyl-3-CH), 7.01-6.88 (m, 3H, phenyl-4-CH, phenyl-5-CH and phenyl-6-CH), 5.33-5.29 (s, 2H, OCH₂O), 3.89-3.83 (m, 2H, CH₃OCH₂), 3.60-3.54 (m, 6H, and 2′- & 6′-CH₂), 3.39-3.36 (m, 3H, OCH₃), 3.03-2.96 (t, J=5.0 Hz, 4H, 3′- & 5′-CH₂), 1.49-1.45 (s, 9H, 3×CH₃).

### 2(iii) 1-(2-((2-methoxyethoxy)methoxy)phenyl)piperazine (4, PG=MEM)

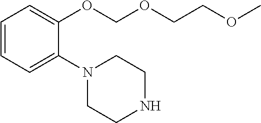 [SMILES: COCCOCOc1ccccc1N1CCNCC1]

tert-Butyl 4-(2-((2-methoxyethoxy)methoxy)phenyl)piperazine-1-carboxylate (900 mg, 2.46 mmol) was slowly dissolved in neat TFA (5 mL) and was stirred at ambient temperature for 10 mins. The reaction mixture was diluted with ether (50 mL) and neutralised with saturated potassium carbonate solution (10 mL) at 0° C. The aqueous layer was washed with diethyl ether (2*50 mL) and the combined organics were dried over magnesium sulfate, filtered and evaporated to dryness to afford a pale yellow residue. The aqueous layer was then basified with additional saturated potassium carbonate solution (5 mL) and the residue was re-dissolved in DCM (10 mL) and partitioned with water and additional DCM (2*30 mL). The organic portion was dried (phase separation cartridge) and evaporated to dryness to afford 1-(2-((2-methoxyethoxy)methoxy)phenyl)piperazine as a pale yellow oil (450 mg, 69%).

¹H NMR (301 MHz, CHLOROFORM-D) δ 7.10-7.03 (m, 1H, phenyl-3-CH), 6.96-6.84 (m, 3H, phenyl-4-CH, phenyl-5-CH and phenyl-6-CH), 5.29-5.23 (s, 2H, OCH₂O), 3.90-3.73 (m, 2H, CH₃OCH₂), 3.60-3.43 (m, 2H, CH₂CH₂OCH₂), 3.40-3.25 (s, 3H, OCH₃), 3.11-2.89 (s, 8H, 4× piperazinyl-NCH₂).

### 2(iv): 2-(4-(2-((2-methoxyethoxy)methoxy)phenyl)piperazin-1-yl)-N-(pyridin-2-yl)acetamide (5, PG=MEM)

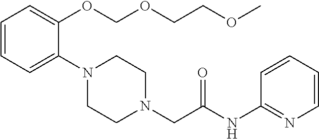 [SMILES: COCCOCOc1ccccc1N1CCN(CC(=O)Nc2ccccn2)CC1]

To a solution of 1-(2-((2-methoxyethoxy)methoxy)phenyl)piperazine (450 mg, 1.69 mmol) in DMF (15 mL) was added potassium carbonate (584 mg, 4.22 mmol) and the mixture stirred at 80° C. for 45 minutes. To the cooled reaction mixture was added 2-chloro-N-(pyridin-2-yl)acetamide 3 (288 mg, 1.69 mmol) and sodium iodide (38 mg, 0.25 mmol) and stirring continued at 80° C. for 3 h. The cooled reaction mixture was evaporated to remove the majority of the DMF and the residue was partitioned between ethyl acetate (50 mL) and water (50 mL). The organic portion was washed with brine (50 mL), dried over magnesium sulfate, filtered and evaporated to dryness and the residue was purified by column chromatography on silica gel eluting with petroleum ether (A): ethyl acetate (B) (40-90% (B), 50 g, 25.0 CV, 40 mL/min) to afford 24442-((2-methoxyethoxy)methoxy)phenyl)piperazin-1-yl)-N-(pyridin-2-yl)acetamide as a pale yellow oil (515 mg, 76%).

¹H NMR (301 MHz, CHLOROFORM-D) δ 9.62-9.56 (s, 1H, NH), 8.29-8.25 (ddd, J=4.9, 2.0, 0.9 Hz, 1H, pyridyl-6-CH), 8.25-8.20 (m, 1H, pyridyl-3-CH), 7.70-7.63 (m, 1H, pyridyl-4-CH), 7.11-6.88 (m, 5H, 4× phenyl-CH and pyridyl-5-CH), 5.29-5.26 (s, 2H, OCH₂O), 3.85-3.79 (m, 2H, CH₃OCH₂), 3.56-3.50 (m, 2H, CH₂CH₂OCH₂), 3.35-3.32 (s, 3H, OCH₃), 3.20-3.11 (m, 6H, 2″-CH₂ and 3′- & 5′-CH₂), 2.81-2.71 (t, J=4.8 Hz, 4H, 2′- & 6′-CH₂). ¹³C NMR (76 MHz, CHLOROFORM-D) δ 169.18 (C═O), 151.08 (phenyl-1-C), 150.10 (pyridyl-2-C), 148.08 (pyridyl-6-CH), 142.14 (phenyl-2-C), 138.39 (pyridyl-4-CH), 123.23 (pyridyl-5-CH), 122.88 (phenyl-6-CH), 119.94 (phenyl-4-CH), 118.82 (phenyl-5-CH), 116.87 (phenyl-3-CH), 113.92 (pyridyl-3-CH), 94.33 (OCH₂O), 71.68 (CH₃OCH₂), 67.99 (CH₂CH₂OCH₂), 62.36 (2″-CH₂), 59.11 (OCH₃), 53.99 (3′- & 5′-CH₂), 50.75 (2′- & 6′-CH₂).

### 2(v). N-(2-(4-(2-((2-methoxyethoxy)methoxy)phenyl)piperazin-1-yl)ethyl)pyridin-2-amine (6, PG=MEM)

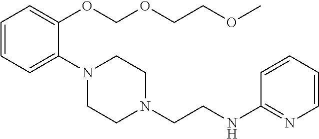 [SMILES: COCCOCOc1ccccc1N1CCN(CCNc2ccccn2)CC1]

To a solution of 2-(4-(2-((2-methoxyethoxy)methoxy)phenyl)piperazin-1-yl)-N-(pyridin-2-yl)acetamide (500 mg, 1.25 mmol) in THF (15 mL) at 0° C. was slowly added LiAlH₄(142 mg, 3.75 mmol, 1.87 mL of a 2.0 M solution in THF) and was stirred at ambient temperature for three hours. The reaction mixture was cooled to 0° C. and quenched with saturated ammonium chloride solution (3 mL) then filtered with ethyl acetate and the resultant solution was partitioned between ethyl acetate (25 mL) and water (25 mL). The organic portion was dried over magnesium sulfate, filtered and evaporated to dryness to afford a yellow oily residue. The residue was purified by column chromatography on silica gel eluting with dichloromethane (A): methanol (B) (2-10% (B), 50 g, 21.2 CV, 40 mL/min) to afford N-(2-(4-(2-((2-methoxyethoxy)methoxy)phenyl)piperazin-1-yl)ethyl)pyridin-2-amine as a yellow oil (195 mg, 40%).

¹H NMR (301 MHz, CHLOROFORM-D) δ 8.16-8.01 (ddd, J=5.1, 1.9, 0.9 Hz, 1H, pyridyl-6-CH), 7.43-7.36 (m, 1H, pyridyl-4-CH), 7.13-7.08 (m, 1H, phenyl-3-CH), 7.01-6.91 (m, 3H, 4-, 5- & 6-phenyl-CH), 6.58-6.52 (ddd, J=7.1, 5.1, 0.9 Hz, 1H, pyridyl-3-CH), 6.43-6.38 (dt, J=8.4, 0.9 Hz, 1H, pyridyl-5-CH), 5.35-5.23 (s, 2H, OCH₂O), 5.18-5.08 (t, J=4.6 Hz, 1H, NH), 3.88-3.82 (m, 2H, CH₃OCH₂), 3.59-3.54 (m, 2H, CH₂CH₂OCH₂), 3.38-3.36 (s, 5H, OCH₃ and 1″-CH₂), 3.12-3.07 (m, 4H, 3′- &5′-CH₂), 2.72-2.62 (m, 6H, 2″-CH₂ and 2′- & 6′-CH₂). ¹³C NMR (76 MHz, CHLOROFORM-D) δ 158.90 (phenyl-1-C), 150.09 (pyridyl-2-C), 148.26 (pyridyl-6-CH), 142.48 (phenyl-2-C), 137.39 (pyridyl-4-CH), 123.00 (phenyl-6-CH), 122.88 (phenyl-4-CH), 118.70 (phenyl-5-CH), 116.89 (phenyl-3-CH), 112.78 (pyridyl-5-CH), 107.15 (pyridyl-3-CH), 94.35 (OCH₂O), 71.71 (CH₃OCH₂), 67.98 (CH₂CH₂OCH₂), 59.13 (OCH₃), 56.89 (2″-CH₂), 53.33 (3′- & 5′-CH₂), 50.74 (2′- & 6′-CH₂), 38.61 (1″-CH₂).

### 2(vi): (1r,4r)-4-((2-(4-(2-((2-methoxyethoxy)methoxy)phenyl)piperazin-1-yl)ethyl)(pyridin-2-yl)carbamoyl)cyclohexanecarboxylic acid (10, PG=MEM)

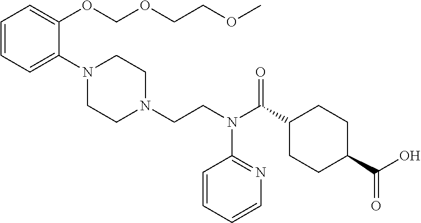

A mixture of trans-1,4-cyclohexanedicarboxlic acid (1 g, 5.813 mmol) and oxalyl chloride (7.4 g, 58.2 mmol, 5 mL) was heated to reflux for 1 h. The excess oxalyl chloride was co-distilled using dichloromethane under nitrogen atmosphere. To a solution of a portion of the 1,4-cyclohexane diacid chloride (120 mg, 0.57 mmol) in DCM (5 mL) was added a solution of N-(2-(4-(2-((2-methoxyethoxy)methoxy)phenyl)piperazin-1-yl)ethyl)pyridin-2-amine (178 mg, 0.46 mmol) and TEA (64 mg, 0.63 mmol, 0.09 mL) in DCM (5 mL) and was stirred at ambient temperature for 1 hour.

The reaction mixture was quenched with water (4 mL) and the organic portion was evaporated to dryness The residue was dissolved in 10% sodium hydroxide solution (1 mL), diluted with water (10 mL) and DCM (10 mL). The organic portion was collected and the aqueous was adjusted to pH 6.5 using conc. HCl and extracted with DCM (2*30 mL) and the combined organic portions were dried (phase sep cartridge) and evaporated to dryness to afford 13 mg of a colourless oil. To the aqueous portion was added diethyl ether (50 mL); the organic portion was dried over magnesium sulfate, filtered, combined with the colourless oil and evaporated to dryness to afford (1s,4s)-4-((2-(4-(2-((2-methoxyethoxy) methoxy)phenyl)piperazin-1-yl)ethyl)(pyridin-2-yl)carbamoyl)cyclohexanecarboxylic acid (240 mg, 77%) in total.

¹H NMR (301 MHz, CHLOROFORM-D) δ 8.57-8.42 (m, 1H, pyridyl-6-CH), 7.82-7.68 (m, 1H, pyridyl-4-CH), 7.32-7.17 (m, 2H, pyridyl-3-CH and pyridyl-5-CH), 7.15-7.03 (m, 1H, phenyl-3-CH), 7.02-6.81 (m, 3H, 3× phenyl-CH), 5.39-5.14 (m, 2H, OCH₂O), 4.05-3.72 (m, 2H, 1″-CH₂), 3.72-3.22 (m, 7H, 2×OCH₂ and OCH₃), 3.02-2.95 (s, 4H, 2× piperazinyl-CH₂), 2.75-2.52 (m, 6H, 2× piperazinyl-CH₂ and 2″-CH₂), 2.34-2.09 (m, 2H, 2× cyclohexyl CH), 2.07-1.68 (m, 4H, 4× cyclohexyl-CHH), 1.68-1.53 (m, 2H, 2× cyclohexyl-CHH), 1.36-1.08 (m, 2H, 2× cyclohexyl′

### 2(vii) (1r,4r)-4-(hydroxymethyl)-N-(2-(4-(2-((2-methoxyethoxy) methoxy)phenyl)piperazin-1-yl)ethyl)-N-(pyridin-2-yl)cyclohexanecarboxamide (12, PG=MEM)

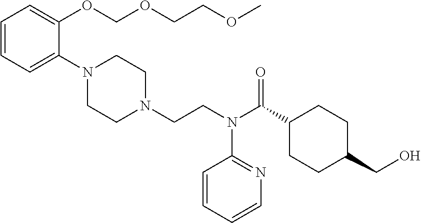

To a solution of (1r,4r)-4-((2-(4-(2-((2-methoxyethoxy)methoxy)phenyl)piperazin-1-yl)ethyl)(pyridin-2-yl)carbamoyl)cyclohexanecarboxylic acid (240 mg, 0.44 mmol) in anhydrous THF (4 mL) at 0° C. was added borane-THF complex (191 mg, 2.22 mmol, 2.22 mL of a 1.0 M solution in TRF) once an hour for three hours. After complete addition, the reaction mixture was stirred at ambient temperature for one hour. The reaction mixture was quenched with water (2 mL) and evaporated. The residue was dissolved in methanol (10 mL) and heated at reflux for one hour. The reaction mixture was evaporated to dryness to afford a colourless solid residue (520 mg) that was insoluble in chloroform and sparingly soluble in methanol. ¹H NMR indicated the presence of a large amount of water so the residue was partitioned between water (20 mL) and diethyl ether (50 mL). The organic portion was dried over magnesium sulfate, filtered and evaporated to dryness. The residue was purified by column chromatography on high performance silica gel eluting with DCM (A): methanol (B) (2-10% (B), 12 g, 28.0 CV, 30 mL/min) to afford (1r,4r)-4-(hydroxymethyl)-N-(2-(4-(2-((2-methoxyethoxy)methoxy)phenyl)piperazin-1-yl)ethyl)-N-(pyridin-2-yl)cyclohexanecarboxamide as a colourless oil (65 mg, 28%).

LC-MS: m/z calcd for C₂₆H₃₆N₄O₃, 526.3, found, 527.3 (M+H)⁺

¹H NMR (301 MHz, CHLOROFORM-D) δ 8.56-8.43 (m, 1H, pyridyl-6-CH), 7.82-7.68 (m, 1H, pyridyl-4-CH), 7.32-7.18 (m, 2H, pyridyl-3-CH and pyridyl-5-CH), 7.00-6.80 (m, 4H, 4× phenyl-CH), 5.28-5.24 (d, J=2.6 Hz, 2H, OCH₂O), 3.87-3.79 (m, 2H, CH₃OCH₂), 3.59-3.52 (m, 2H, CH₂CH₂OCH₂), 3.38-3.35 (m, 5H, OCH₃ and 1″-CH₂), 3.00-2.93 (s, 4H, 3′- & 5′-CH₂), 2.63-2.49 (m, 6H, 2″-CH₂ and 2′- & 6′-CH₂), 1.88-1.70 (m, 4H, 4× cyclohexyl-CHH), 1.70-1.21 (m, 4H, 4× cyclohexyl-CHH), 1.06-0.83 (m, 1H, cyclohexyl-CH), 0.83-0.64 (m, 1H, cyclohexyl-CH).

¹³C NMR (76 MHz, CHLOROFORM-D) δ 176.03 (C═O), 150.03 (pyridyl-6-CH), 149.22 (pyridyl-2-C), 142.37 (phenyl-1-C), 138.29 (phenyl-2-C), 138.12 (pyridyl-4-CH), 122.89 (pyridyl-3-CH), 122.81 (pyridyl-5-CH), 122.32 (phenyl-6-CH), 118.55 (phenyl-4-CH), 116.87 (phenyl-5-CH), 111.18 (phenyl-3-CH), 94.31 (OCH₂O), 71.69 (CH₃OCH₇), 68.34 (CH₂CH₂OCH₂), 67.95 (CH₂OH), 59.14 (OCH₃), 53.55 (3′- & 5′-CH₂), 50.70 (2′- & 6′-CH₂), 42.47 (cyclohexyl-CHC(═O)N), 39.70 (cyclohexyl-CH(CH₂OH), 33.63 (1″-CH₂), 29.01 (2× cyclohexyl-CH₂(CHC(═O)N)), 28.57 (2× cyclohexyl-CH₂(CHCH₂OH)).

### 2(viii) (1r,4r)-4-(fluoromethyl)-N-(2-(4-(24(2-methoxyethoxy) methoxy)phenyl)piperazin-1-yl)ethyl)-N-(pyridin-2-yl)cyclohexanecarboxamide

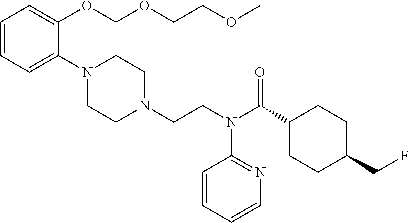

To a solution of (1r,4r)-4-(hydroxymethyl)-N-(2-(4-(2-((2-methoxyethoxy)methoxy) phenyl)piperazin-1-yl)ethyl)-N-(pyridin-2-yl)cyclohexanecarboxamide (65 mg, 0.12 mmol) in DCM (5 mL) in an ice-water bath was added DAST (40 mg, 0.25 mmol, 32 uL) and the solution was stirred at ambient temperature for 23 hours. The reaction mixture was quenched with 10% aqueous sodium bicarbonate solution (10 mL) and partitioned between the aqueous and DCM (20 mL). The organic portion was dried (phase separation cartridge) and evaporated to dryness. The residue was purified by column chromatography on high performance silica gel eluting with DCM (A): methanol (B) (2-10% (B), 12 g, 28.0 CV, 30 mL/min) to afford (1r,4r)-4-(fluoromethyl)-N-(2-(4-(2-((2-methoxyethoxy)methoxy)phenyl)piperazin-1-yl)ethyl)-N-(pyridin-2-yl)cyclohexanecarboxamide as a colourless solid (7 mg).

### 2(ix) (1r,4r)-4-(fluoromethyl)-N-(2-(4-(2-hydroxyphenyl)piperazin-1-yl)ethyl)-N-(pyridin-2-yl)cyclohexanecarboxamide

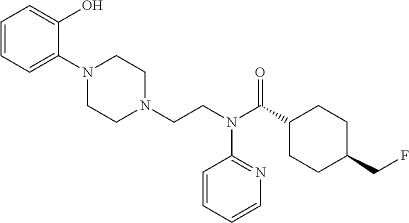

To a solution of (1r,4r)-4-(fluoromethyl)-N-(2-(4-(2-((2-methoxyethoxy)methoxy) phenyl)piperazin-1-yl)ethyl)-N-(pyridin-2-yl)cyclohexanecarboxamide (7 mg, 13.2 umol) in DCM (1 mL) was added TFA (0.5 mL) and the solution stirred at ambient temperature for 4 days. The reaction mixture was quenched with saturated potassium carbonate solution and partitioned between DCM (10 mL) and water (10 mL); the organic portion was dried (phase separation cartridge) and evaporated to dryness. The residue was purified by column chromatography on silica gel eluting with DCM (A): methanol (B) (3% (B), 4 g, 30.0 CV, 18 mL/min) to afford (1r,4r)-4-(fluoromethyl)-N-(2-(4-(2-hydroxyphenyl)piperazin-1-yl)ethyl)-N-(pyridin-2-yl)cyclohexanecarboxamide (2 mg)

LC-MS: m/z calcd for C₂₅H₃₃FN₄O₂, 440.3; found, 441.3 (M+H)⁺

### Example 3

### Synthesis of (1r,4r)-4-([¹⁸F]fluoromethyl)-N-(2-(4-(2-hydroxyphenyl)piperazin-1-yl)ethyl)-N-(pyridin-2-yl)cyclohexanecarboxamide

### 3(i) ((1r,4r)-4-((2-(4-(2-((2-methoxyethoxy)methoxy)phenyl)piperazin-1-yl)ethyl)(pyridin-2-yl)carbamoyl)cyclohexyl)methyl 4-methylbenzenesulfonate

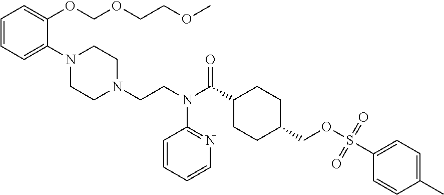

To a solution of (1r,4r)-4-(fluoromethyl)-N-(2-(4-(2-((2-methoxyethoxy)methoxy)phenyl)piperazin-1-yl)ethyl)-N-(pyridin-2-yl)cyclohexanecarboxamide (100 mg, 0.19 mmol) in DCM (5 mL) is added tosyl chloride (59 mg, 0.28 mmol) and TEA (5 drops). The mixture is stirred at 25° C. for 24 h. The reaction mixture is quenched with 10% aqueous sodium bicarbonate solution (5 mL) and the DCM layer separated, dried over sodium sulfate and evaporated to dryness. The residue is purified by column chromatography on neutral alumina (100 g) and eluting with hexane (A): ethyl acetate (B) (10-50% (B), to afford ((1r,4r)-4-((2-(4-(2-((2-methoxyethoxy)methoxy) phenyl)piperazin-1-yl)ethyl)(pyridin-2-yl)carbamoyl)cyclohexyl)methyl 4-methylbenzenesulfonate. Deprotection to remove the protecting group on the hydroxyl may be carried out by acid hydrolysis either before or after the radiolabelling step 3(ii).

### 3(ii) (1r,4r)-4-([¹⁸F]fluoromethyl)-N-(2-(4-(2-hydroxyphenyl)piperazin-1-yl)ethyl)-N-(pyridin-2-yl)cyclohexanecarboxamide

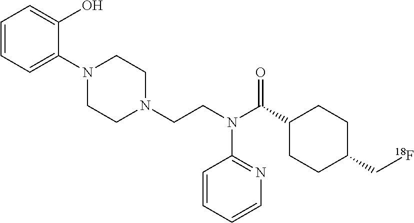

Potassium carbonate solution (50 μL, 0.1 M) is added to kryptofix (5.0 mg) and anhydrous acetonitrile*(0.50 mL) in a 3 mL Wheaton vial equipped with a stirrer vane. [¹⁸F]fluoride (aq.) is added to the vial, and heated to 110° C. under a stream of N₂ to azeotropically dry the [¹⁸F]fluoride. Two further portions of anhydrous acetonitrile (2×0.5 mL) are added and similarly dried. The reaction vial is cooled to room temperature, and the precursor ((1r,4r)-4-((2-(4-(2-hydroxyphenyl)piperazin-1-yl)ethyl)(pyridin-2-yl)carbamoyl)cyclohexyl)methyl 4-methylbenzenesulfonate (1.0 mg) in anhydrous DMF (150 μL) is added. The reaction is stirred at 110° C. for 30 min. The reaction is diluted with acetonitrile (0.6 mL) and water (1.0 mL) and loaded to a semi-preparative HPLC system. The product is collected using a manual switch, diluted with water to a total volume of 20 mL, and loaded onto a tC 18 Light Sep-pak cartridge (primed with 1 mL ethanol and 2 mL water). The product is eluted with ethanol (0.5 mL) and diluted with phosphate buffered saline (4.5 mL).

### Example 4

### Synthesis of (1s,41s)-4-(fluoromethyl)-N-(2-(4-(2-methoxyphenyl)-piperazin-1-yl)ethyl)-N-(pyridin-2-yl)cyclohexanecarboxamide

### 4(i) (1s,4s)-4-((2-(4-(2-methoxyphenyl)piperazin-1-yl)ethyl)(pyridin-2-yl)carbamoyl)cyclohexanecarboxylic acid

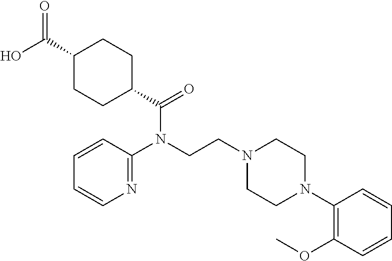 [SMILES: COc1ccccc1N1CCN(CCN(c2ccccn2)C(=O)[C@H]2CC[C@@H](C(=O)O)CC2)CC1]

A mixture of N-(2-(4-(2-methoxyphenyl)piperazin-1-yl)ethyl)pyridin-2-amine (0.9 g, 2.88 mmol) and triethylamine (0.58 g, 5.81 mmol, 0.81 ml) dissolved in DCM (15 ml) and was slowly added to (1s,4s)-cyclohexane-1,4-dicarbonyl dichloride in DCM at 0° C. for 1 h under a dry nitrogen atmosphere. The reaction mixture was stirred for 2 h at room temperature before it was cooled to 0° C. and acidified to pH 2, using concentrated HCl. The DCM layer was separated out. The aqueous layer was then neutralized with solid sodium bicarbonate and the product that precipitated out was extracted into DCM. The DCM layer was dried over anhydrous sodium sulfate and evaporated to obtain crude (1s,4s)-4-((2-(4-(2-methoxyphenyl)piperazin-1-yl)ethyl)(pyridin-2-yl)carbamoyl)cyclohexanecarboxylic acid (1.4 g). The product was used directly in the next step with no further purification.

LC-MS. m/z calcd for C₂₆H₃₄N₄O₄, 466.3; found, 466.2 (M)⁺.

Reduction and fluorination were carried out under the same conditions as described in Example 1 for the trans-isomer.

### Comparative Example 5

### Prior Art Reduction of (1s,4s-methyl 4-((2-(4-(2-methoxyphenyl)piperazin-1-yl)ethyl)-N-(pyridin-2-yl)cyclohexanecarboxamide to (1s,4s)-4-(hydroxymethyl)-N-(2-(4-(2-methoxyphenyl)piperazin-1-yl)ethyl)-N-(pyridin-2-yl)cyclohexanecarcoxamide

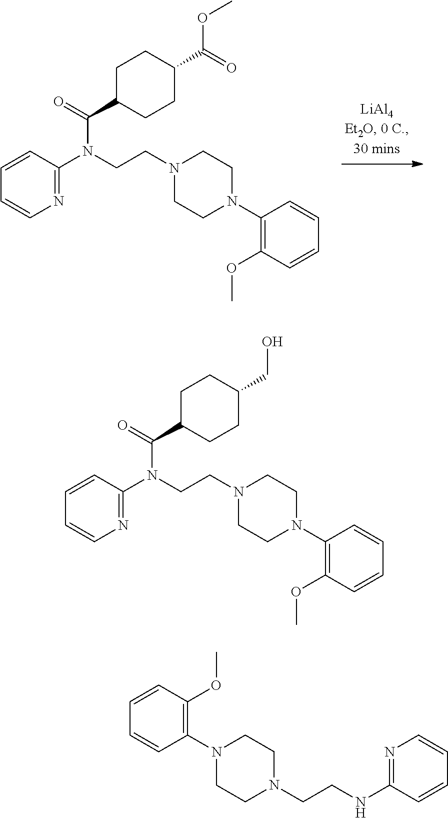

To a solution of (1s,4s)-methyl 4-((2-(4-(2-methoxyphenyl)piperazin-1-yl)ethyl)(pyridin-2-yl)carbamoyl)cyclohexanecarboxylate (1.35 g, 2.81 mmol) in diethyl ether (25 mL) at 0° C. was added lithium aluminium hydride (2.95 mL of a 1.0M solution in ether, 2.95 mmol) and the solution stirred at 0° C. for 30 mins under a nitrogen atmosphere. The reaction mixture was quenched with saturated ammonium chloride solution (30 mL), partitioned with diethyl ether (20 mL) and the organic portion was dried over anhydrous magnesium sulfate, filtered and evaporated to dryness.

The residue was purified by column chromatography on high performance silica gel eluting with DCM (A): methanol (B) (5-10% (B), 50 g, 24.3 CV, 40 mL/min) to afford a 60:40 mixture of the (1s,4s)-4-(hydroxymethyl)-N-(2-(4-(2-methoxyphenyl)piperazin-1-yl)ethyl)-N-(pyridin-2-yl)cyclohexanecarboxamide and N-(2-(4-(2-methoxyphenyl)piperazin-1-yl)ethyl)pyridin-2-amine. Further chromatography was carried out but it was not possible effectively to separate the products.

What is claimed is:

1. A method of making a compound of Formula I: 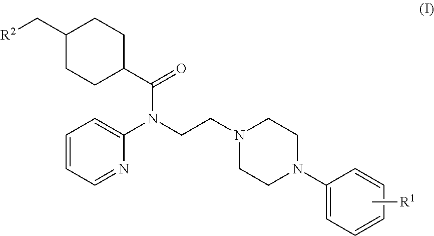
wherein:
R¹ is hydrogen, hydroxy, halogen or C₁₋₄ alkoxy;
R² is hydrogen, fluoro, bromo, chloro, C₁₋₄ alkyl, or is a leaving group;
wherein said compound optionally comprises one atom detectable in an in vivo imaging method;
or a pharmaceutically acceptable salt thereof, wherein said method comprises:
(i) borane reduction of a compound of Formula II: 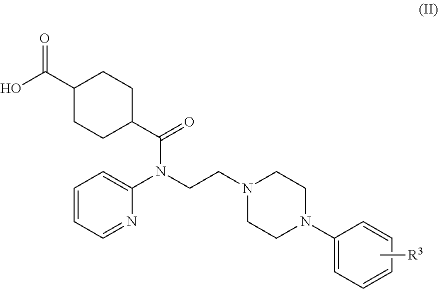
wherein R³ is as defined for R¹;
to obtain a compound of Formula III: 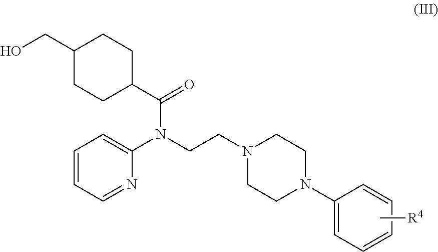
wherein R⁴ is as defined for R¹
(ii) conversion of said compound of Formula III to obtain said compound of Formula I.

2. The method of claim 1 wherein said compound of Formula I is of Formula I-trans: 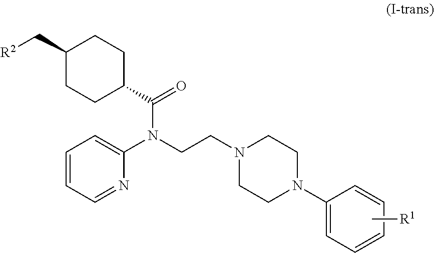
said compound of Formula II is of Formula II-trans: 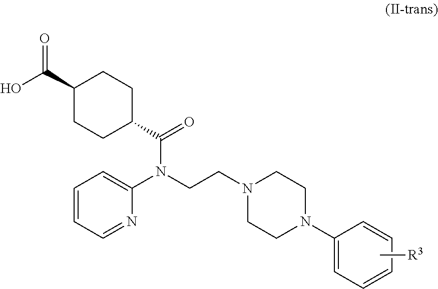
and said compound of Formula III is of Formula III-trans: 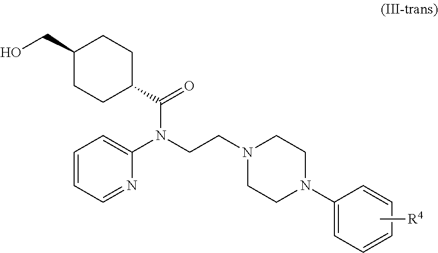
wherein R¹⁻⁴ are as defined in claim 1.

3. The method of claim 1 wherein said compound of Formula I is of Formula I-cis: 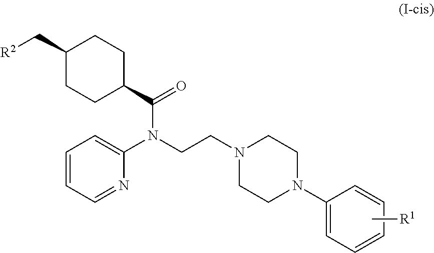
said compound of Formula II is of Formula II-cis: 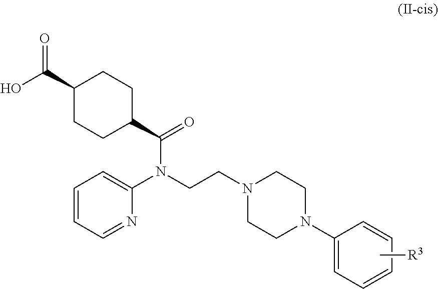
and said compound of Formula III is of Formula III-cis: 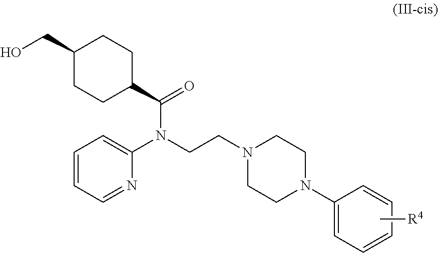
wherein R¹⁻⁴ are as defined in claim 1.

4. The method of claim 1 wherein R¹ is hydroxyl.

5. The method of claim 1 wherein R¹ is methoxy.

6. The method of claim 1 wherein said compound of Formula I comprises an atom detectable in an in vivo imaging method.

7. The method of claim 6 wherein said atom detectable in an in vivo imaging method is ¹⁸F.

8. The method of claim 7 wherein R² is ¹⁸F.

9. The method of claim 1 wherein said borane reduction step is carried out using a reagent comprising diborane (B₂H₆) or a Lewis acid-Lewis base complex of borane (BH₃).

10. The method of claim 9 wherein said Lewis acid-Lewis base complex of BH₃ comprises BH₃.THF (tetrahydrofuran), or BH₃.Me₂S (dimethylsulfide).

11. The method claim 1 wherein said compound of Formula II is obtained by acid hydrolysis of a compound of Formula IIa: 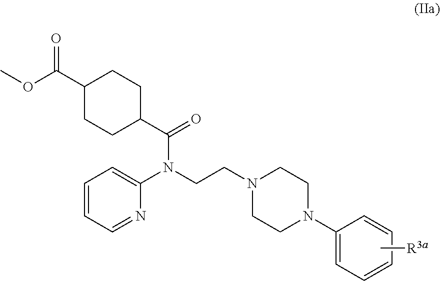
wherein R^{3a} is hydrogen, hydroxy, halogen or C₁₋₄ alkoxy.

12. The method of claim 11 wherein said compound of Formula IIa is a compound of Formula IIa-trans:

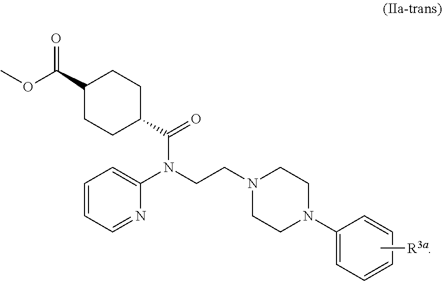

13. The method of claim 11 wherein said compound of Formula IIa is a compound of Formula IIa-cis:

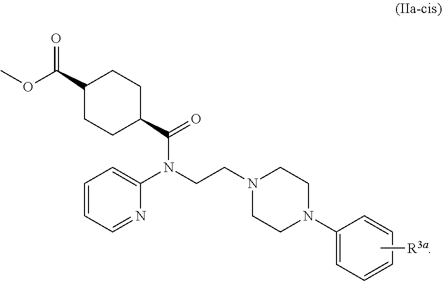

14. The method of claim 1 wherein said compound of Formula II is obtained by reacting a compound of Formula IIb: 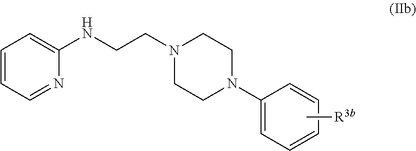
wherein R^{3b} is hydrogen, hydroxy, halogen or C₁₋₄ alkoxy;
with an excess of cyclohexane-1,4-dicarboxylic acid in the presence of oxalyl chloride.

15. The method of claim 14 wherein said cyclohexane-1,4-dicarboxylic acid is trans-cyclohexane-1,4-dicarboxylic acid.

16. The method of claim 14 wherein said cyclohexane-1,4-dicarboxylic acid is cis-cyclohexane-1,4-dicarboxylic acid.

17. The method of claim 1 wherein said conversion step comprises reaction of said compound of Formula III with a suitable source of a halogen to obtain a compound of Formula I wherein R² is halogen.

18. The method of claim 17 which further comprises formulation of said compound of Formula I to obtain a pharmaceutical composition.

19. The method as defined in of claim 1 wherein said conversion step comprises reaction of said compound of Formula III with a suitable source of a leaving group to obtain a compound of Formula I wherein R² is a leaving group.

20. The method of claim 19 which comprises the further step of reacting said compound of Formula I wherein R² is a leaving group with a suitable source of ¹⁸F to obtain a compound of Formula I wherein R² is ¹⁸F.

21. The method of claim 20 wherein said suitable source of ¹⁸F is a source of [¹⁸F]fluoride (¹⁸F).

22. The method of claim 20 wherein said reacting is automated.

23. The method of claim 20 wherein said reacting is carried out on an automated synthesis apparatus.

24. The method of claim 20 which further comprises formulation of said compound of Formula I to obtain a radiopharmaceutical composition.

25. A method of making a compound of Formula III of claim 1 wherein said method comprises the step of borane reduction of a compound of Formula II: 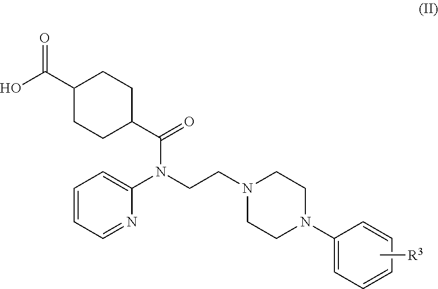
wherein R³ is hydrogen, hydroxy, halogen or C₁₋₄ alkoxy.
